(19) United States
(12) Patent Application Publication
(10) Pub. No.: US 2021/0330182 A1
(43) Pub. Date: Oct. 28, 2021

(54) OPTICAL COHERENCE TOMOGRAPHY IMAGE PROCESSING METHOD

(71) Applicant: WENZHOU MEDICAL UNIVERSITY, Wenzhou District, Zhejiang (CN)

(72) Inventors: Yune ZHAO, Wenzhou City (CN); Jinhai HUANG, Wenzhou City (CN); Hang YU, Wenzhou City (CN)

(21) Appl. No.: 16/613,379

(22) PCT Filed: Aug. 7, 2018
(86) PCT No.: PCT/CN2018/099142

§ 371 Date: Nov. 13, 2019

(51) Int. Cl. A61B 3/10; G06T 7/00

(52) U.S. Cl. CPC A61B 3/102; G06T 2207/10101; G06T 2207/30041; G06T 7/0012

(57) ABSTRACT

The present invention discloses an optical coherence tomography image processing method, comprising the following steps: step 1: collecting anterior segment tomography images by means of an optical coherence tomography technology, to obtain an anterior segment black-and-white image; step 2: performing pseudo-coloring processing and color space conversion on the anterior segment black-and-white image and performing superposed threshold binarization, to differentiate cornea, iris and crystalline lens potential areas, and then obtaining cornea, iris and crystalline lens areas separately by means of blob shape analyses; step 3: performing, by means of tectonics operations, spot filling and collapse processing on the cornea, iris and crystalline lens areas obtained in step 2; and step 4: performing boundary tracing on the image by means of a level set algorithm, to precisely trace surface boundaries of all parts of an anterior segment and find the anterior segment. According to the present invention, the bottleneck of a low speed resulted from applying the level set algorithm to the whole image is overcome, real-time extraction and analysis of features of the anterior segment tomography image are implemented, and reliable basic data is provided for subsequent obtaining of anterior segment clinical parameters.

### FIELD OF THE INVENTION

The present invention relates to an ophthalmology image processing method, and in particular to an optical coherence tomography image processing method.

### DESCRIPTION OF THE PRIOR ART

There are increasingly more eye problems because of a growing number of electronic products and the increasingly serious aging problem. New ophthalmology imaging technologies develop constantly; therefore, ophthalmologists can observe eyes more directly and the diagnosis rate is also increased significantly. The computer-assisted diagnostic technology mainly focuses on how to efficiently process various kinds of ophthalmology imaging information by means of the image processing technology, so as to assist the ophthalmologists in diagnosing and even in making surgery plans. Therefore, the computer-assisted diagnostic technology has a broad application prospect and a significant social effect.

The anterior segment is a part of an eyeball, and specifically includes: the whole cornea, iris, ciliary body, anterior chamber, posterior chamber, crystalline lens suspensory ligament, chamber angle, parts of the crystalline lens, surrounding vitreous body, retina, extraocular muscle attachment site, conjunctiva and the like. Anterior segment image analysis and processing are of great importance to eye disease determination. The medical image processing technology is an intersection of various subjects such as medicine, mathematics, and computer, and develops constantly as a key of the computer-assisted diagnosis. As the ophthalmic medicine flourishes, people propose increasingly high requirements for the ophthalmology image processing and analysis, and a further study on medical image processing and analysis is of great significance.

Therefore, a person skilled in the art dedicates to developing a machine vision-based anterior segment tomography image feature extraction method, i.e., an optical coherence tomography image processing method. In the prior art, a level set algorithm is generally adopted for the whole image, and the bottleneck of a low speed cannot be overcome.

### SUMMARY OF THE INVENTION

In view of the defects of the prior art, the technical problem to be solved by the present invention is to provide an optical coherence tomography image processing method, comprising the following steps: step 1: collecting anterior segment tomography images by means of an optical coherence tomography technology, to obtain an anterior segment black-and-white image; step 2: performing pseudo-coloring processing and color space conversion on the anterior segment black-and-white image obtained in step 1, so that distances between color distributions of a cornea, an iris, and a crystalline lens in the converted image are maximized, then performing superposed threshold binarization on the image which has been subject to the color space conversion, to differentiate a cornea potential area, an iris potential area, and a crystalline lens potential area, and then eliminating noise and interference areas separately by means of blob shape analyses, to obtain a cornea area, an iris area, and a crystalline lens area; step 3: performing, by means of tectonics operations, spot filling and collapse processing on the cornea area, the iris area, and the crystalline lens area obtained in step 2; and step 4: performing, by means of a level set algorithm, boundary tracing on the image processed by step 3, to precisely trace surface boundaries of all parts of an anterior segment and find the anterior segment.

Further, the color space conversion in step 2 uses an L*U*V* color space model; in the L*U*V* color space model, three components are used to represent colors: L* represents image brightness, U* and V* separately represent color differences, a color distance between different colors is defined by the Euclidean distance, which is shown by the following formula:

Δd=\sqrt{(Lₐ*−L_{b}*)²+(Uₐ*−U_{b}*)²+(Vₐ*−V_{b}*)²}

wherein a and b separately represent two points in the image, either point has three components: L*, U*, and V*, the two points are respectively represented as Lₐ* , Uₐ*, Vₐ* or L_{b}*, U_{b}*, V_{b}*, and Δd represents a color distance between a and b.

Further, the superposed threshold binarization in step 2 requires 3n threshold spaces, and n≥1. In order to preliminarily isolate the cornea potential area, the crystalline lens potential area, and the iris potential area, 3n threshold spaces are required, and n≥1.

Further, the blob shape analyses for the cornea potential area, the iris potential area, and the crystalline lens potential area in step 2 separately need to be performed n times, i.e., the blob shape analysis needs to be performed once for a differentiated potential area each time after the multi-threshold binarization is performed. Because there are three potential areas: the cornea potential area, the iris potential area, and the crystalline lens potential area, the multi-threshold binarization requires 3n thresholds, and n≥1. Moreover, the blob shape analyses for the cornea potential area, the iris potential area, and the crystalline lens potential area separately need to be performed n times.

Further, the surface boundaries of all parts of the anterior segment in step 4 refer to: cornea front surface, cornea rear surface, iris front surface, and crystalline lens front surface boundaries.

Further, the anterior segment tomography image is in a bmp or jpeg format.

Further, the anterior segment tomography images collected in step 1 can be of the same resolution or different resolutions.

### Technical Effects

The spot filling and collapse processing are used to expedite the level set algorithm. The level set algorithm requires an enclosed contour. If there is a hole, it is equivalent to that there is an extra contour, which will reduce a speed of the subsequent level set algorithm. The hole filling and expansion processing provide an initial contour for the level set algorithm, thereby improving the speed. Adopting the level set algorithm for the whole image may result in a rather low speed without this initial contour.

According to the present invention, on the premise that a rough contour of an interested area is obtained, the level set algorithm is used to precisely trace an image contour by using the rough contour as an initial level line, thereby overcoming the bottleneck of a low speed resulted from adopting the level set algorithm for the whole image, implementing real-time extraction and analysis of features of an anterior segment tomography image, and providing reliable basic data for subsequent obtaining of anterior segment clinical parameters.

The concept, specific structure, and produced technical effects of the present invention will be further described below with reference to the drawings, so as to fully understand the purposes, features and effects of the present invention.

### BRIEF DESCRIPTION OF THE DRAWINGS

FIG. 1 is a flowchart of a preferred embodiment of an optical coherence tomography image processing method to which the present invention relates.

### DETAILED DESCRIPTION OF THE PREFERRED EMBODIMENTS

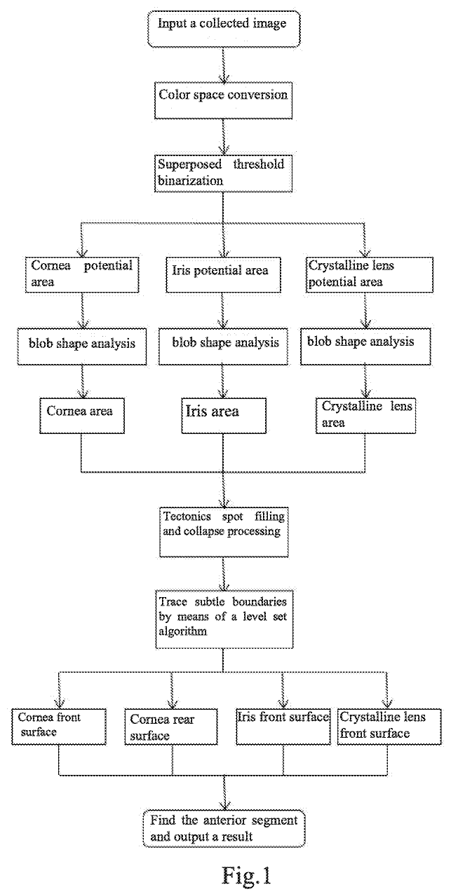

FIG. 1 illustrates an embodiment of the present invention. In this embodiment, a process of an optical coherence tomography image processing method is: first, anterior segment tomography images are collected by means of an optical coherence tomography technology; the collected images are inputted into a computer and stored in a file format of a relatively low compression ratio, for example, a bmp or jpeg format, to ensure that an image has more local details, wherein the images can be of the same resolution or different resolutions; and anterior segment black-and-white images are obtained.

Then, color space conversion is performed on the anterior segment black-and-white image, so that distances between color distributions of a cornea, an iris, and a crystalline lens in the converted image are maximized; next, superposed threshold binarization is performed on the image to differentiate a cornea potential area, an iris potential area, and a crystalline lens potential area; and noise and interference areas of the cornea potential area, the iris potential area, and the crystalline lens potential area are eliminated separately by means of blob shape analyses, to obtain a cornea area, an iris area, and a crystalline lens area.

The color space conversion uses an L*U*V* color space model; in the L*U*V* color space model, three components are used to represent colors: L* represents image brightness, U* and V* separately represent color differences, a color distance between different colors can be defined by the Euclidean distance, which is shown by the following formula:

Δd=\sqrt{(Lₐ*−L_{b}*)²+(Uₐ*−U_{b}*)²+(Vₐ*−V_{b}*)²}

wherein a and b separately represent two points in the image, either point has three components: L*, U*, and V*, the two points are respectively represented as Lₐ*, Uₐ*, Vₐ* or L_{b}*, U_{b}*, V_{b}*, and Δd represents a color distance between a and b.

Therefore, in the color space, a color difference between close points is small, and a color difference between distant points is large. On such a basis, the cornea potential area, the crystalline lens potential area, and the iris potential area can be roughly isolated by means of multi-threshold image segmentation that is provided with prior knowledge.

In addition, in order to preliminarily isolate the cornea potential area, the crystalline lens potential area, and the iris potential area, the multi-threshold binarization requires 3n thresholds, and n≥1. Afterwards, the blob shape analyses for the cornea potential area, the iris potential area, and the crystalline lens potential area separately need to be performed n times.

And then, spot filling and collapse processing are performed on the cornea area, the iris area, and the crystalline lens area by means of tectonics operations. Finally, boundary tracing is performed by means of a level set algorithm, to precisely trace subtle boundaries (comprising cornea front surface, cornea rear surface, iris front surface, and crystalline lens front surface boundaries) of surfaces of all parts of an anterior segment and find the anterior segment, and a result is outputted from the computer.

The preferred specific embodiments of the present invention have already been described above in detail. It shall be understood that one skilled in the art could make various modifications and variations according to the concept of the present invention without contributing any inventive labor. Therefore, any technical solution that could be obtained by one skilled in the art through logical analysis, reasoning or limited experiments according to the concept of the present invention based on the prior art shall be all included in the protection scope defined by the claims.

1. An optical coherence tomography image processing method, comprising the following steps:
step 1: collecting anterior segment tomography images by means of an optical coherence tomography technology, to obtain an anterior segment black-and-white image;
step 2: performing pseudo-coloring processing and color space conversion on the anterior segment black-and-white image obtained in step 1, so that distances between color distributions of a cornea, an iris, and a crystalline lens in the converted image are maximized, then performing superposed threshold binarization on the image which has been subject to the color space conversion, to differentiate a cornea potential area, an iris potential area, and a crystalline lens potential area, and then eliminating noise and interference areas separately by means of blob shape analyses, to obtain a cornea area, an iris area, and a crystalline lens area;
step 3: performing, by means of tectonics operations, spot filling and collapse processing on the cornea area, the iris area and the crystalline lens area obtained in step 2; and
step 4: performing, by means of a level set algorithm, boundary tracing on the image processed by step 3, to precisely trace surface boundaries of all parts of an anterior segment and find the anterior segment.

2. The optical coherence tomography image processing method according to claim 1, wherein the color space conversion in step 2 uses an L*U*V* color space model; in the L*U*V* color space model, three components are used to represent colors: L* represents image brightness, U* and V* separately represent color differences, a color distance between different colors is defined by the Euclidean distance, which is shown by the following formula:
Δd=\sqrt{(Lₐ*−L_{b}*)²+(Uₐ*−U_{b}*)²+(Vₐ*−V_{b}*)²}
wherein a and b separately represent two points in the image, either point has three components: L*, U*, and V*, the two points are respectively represented as Lₐ*, Uₐ*, Vₐ* and L_{b}*, U_{b}*, V_{b}*; and Δd represents a color distance between a and b.

3. The optical coherence tomography image processing method according to claim 1, wherein the superposed threshold binarization in step 2 requires 3n threshold spaces, and n≥1.

4. The optical coherence tomography image processing method according to claim 3, wherein the blob shape analyses for the cornea potential area, the iris potential area, and the crystalline lens potential area in step 2 separately need to be performed n times.

5. The optical coherence tomography image processing method according to claim 1, wherein the surface boundaries of all parts of the anterior segment in step 4 refer to: cornea front surface, cornea rear surface, iris front surface, and crystalline lens front surface boundaries.

6. The optical coherence tomography image processing method according to claim 1, wherein the anterior segment tomography image is in a bmp or jpeg format.

7. The optical coherence tomography image processing method according to claim 1, wherein the anterior segment tomography images collected in step 1 can be of the same resolution or different resolutions.
